(19) United States
(12) Patent Application Publication
(10) Pub. No.: US 2009/0326521 A1
(43) Pub. Date: Dec. 31, 2009

(54) Apparatus and method for treating corneal neovascularization or blood vessel accumulation on the conjunctiva

(76) Inventor: Jaouad Zemmouri, Hem (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/586,284

(22) PCT Filed: Jan. 10, 2005
(86) PCT No.: PCT/EP05/00127

§ 371 Date: Aug. 12, 2009

(30) Foreign Application Priority Data
Jan. 14, 2004 (FR) 0400286

(51) Int. Cl. A61F 9/008

(52) U.S. Cl. 606/4

(57) ABSTRACT

An apparatus for treating corneal neovascularization or blood vessel accumulation on the conjunctiva, which includes a therapeutic light source for emitting a therapeutic light beam at a wavelength between 1.2 μm and 1.3 μm. The light source is preferably a laser for emitting a pulsed beam.

This invention relates to the laser treatment of corneal neovascularization or the accumulation of blood vessels on the conjunctiva.

### PRIOR ART

### Cornea

The cornea is embedded in the anterior opening of the sclera and consists of five layers. The border between the cornea and the sclera is called the limbus, and constitutes a semitransparent zone that has the specificity of adhering to the conjunctiva, a thin membrane that covers the inner surface of the eyelids and the anterior portion of the sclera.

The cornea forms the main lens of the ocular system. For this tissue to be capable of properly performing its function, it must be transparent. Thus, the cornea is normally nonvascularized. By contrast with the cornea, the limbus is rich with nerves and vessels.

### Neovascularization of the Cornea

A number of causes can contribute to the formation of neovessels in the cornea. In general, it can be said that the neovascularization of the cornea results from a sort of call for help by cornea tissue in distress.

One of the main causes is the wearing of corneal lenses (soft or hard).

There are also a number of other causes: infections, allergies, herpes, anoxia, reactions to toxic agents, and so on.

The role of individual susceptibility has also been demonstrated. Indeed, patients suffering from acne rosacea, AIDS, or those who have undergone a radial keratotomy or a penetrating keratoplasty, for example, all have more sensitive corneas, and are therefore more susceptible to the risk of developing neovessels, in particular when wearing lenses.

Regardless of the cause, the formation of these neovessels is detrimental to the transparency of the cornea, and therefore to its function as a lens for the ocular system. Depending on their location and their degree of development, these neovessels can result in a loss of visual acuity. It is therefore essential to be capable of treating an abnormally-vascularized cornea, so as to cause these neovessels to regress, and if possible to make them disappear entirely.

### Treatment Methods

A number of methods for treating neovascularization have been proposed to this day.

A first known method is laser photocoagulation, which was first proposed in the early 1970s. For optimal efficacy, the laser used must have a wavelength of 577 nm. Indeed, one of the hemoglobin absorption peaks is located very precisely at this wavelength. Nevertheless, this laser photocoagulation treatment method, while constituting an effective therapeutic approach, has certain disadvantages.

The goal of laser photocoagulation is to burn the neovessels by means of a thermal laser, causing significant heat to be released in the region of the neovessels. This heat release can detrimentally cause collateral effects on the ocular system.

The known laser, and the most commonly used with the aforementioned wavelength of 577 nm, is a dye laser. However, the costs of acquisition and maintenance of a dye laser are very high, and make this type of machine inaccessible to almost all ophthalmologic centers.

Another treatment method consists of using exogenous chromophores such as Rose Bengal in combination with the Argon laser. The idea is to be capable of using a laser commonly used in ophthalmology offices. Unfortunately, this method requires the intravenous injection of a dye (Rose Bengal) and presents regulatory problems, which, to the knowledge of the applicants, have not been solved.

More recently, photodynamic therapy (PDT) has been proposed, which generally consists of combining a photosensitive drug and a “non-thermal” laser, by contrast with the lasers used in photocoagulation. In particular, the use of a photosensitive drug marketed under the brand name Visudyne® has been proposed. However, to the knowledge of the applicants, this drug has not yet received approval for this use. Moreover, the intravenous injection of this type of drug presents certain disadvantages: it detrimentally causes temporary photosensitization of the patient, which photosensitization requires the patient to avoid any sun exposure for a relatively long period (typically on the order of 48 h); in some patients, the injection of a photosensitive drug can cause adverse effects.

Furthermore, for all of these techniques, the wavelength used is precisely that at which the retina is most sensitive, which presents a danger for the retina.

### The Conjunctiva

The bulbar and palpebral conjunctiva is normally vascularized. However, the excessive accumulation of blood vessels on the conjunctiva is aesthetically undesirable. The accumulation of vessels may result in an increase in the diameter of the vessels and/or an increase in the number of vessels on the conjunctiva. In the case of excessive accumulation that is aesthetically undesirable, it is necessary to treat these blood vessels. To this day, the most widespread method consists of instilling drops of a vasoconstrictor into the eye.

### OBJECTIVES OF THE INVENTION

The main objective of the invention is to propose a new apparatus and a new method for treating corneal neovascularization or the excessive accumulation of vessels on the conjunctiva.

More specifically, another objective of the invention is to propose a new solution to the treatment of corneal neovascularization or the excessive accumulation of vessels on the conjunctiva, which, by contrast with laser photocoagulation, does not cause excessive and destructive heating.

More specifically, another objective of the invention is to propose a new solution for the treatment of corneal neovascularization or the excessive accumulation of vessels on the conjunctiva, which does not require the administration of a product (dye, photosensitive drug, vasoconstrictor).

More specifically, another objective of the invention is to propose a new apparatus for the treatment of corneal neovascularization or the excessive accumulation of vessels on the conjunctiva, which is easy an inexpensive to maintain and/or that is small.

### SUMMARY OF THE INVENTION

All or some of the aforementioned objectives are achieved by the invention, which relates to a new apparatus and a new method for the treatment of corneal neovascularization or the excessive accumulation of vessels on the conjunctiva.

The apparatus of the invention for the treatment of corneal neovascularization or the accumulation of vessels on the conjunctiva comprises a therapeutic light source that is designed to emit a therapeutic light beam having a wavelength between 1.2 μm and 1.3 μm.

The invention also relates to a method for treatment of neovascularization of a cornea or the accumulation of vessels on the conjunctiva, in which the cornea or the limbus is illuminated in the case of corneal neovascularization. or the conjunctiva is illuminated in the case of the accumulation of vessels on the conjunctiva, with a therapeutic light beam having a wavelength between 1.2 μm and 1.3 μm, preferably without the prior administration of a product, and in particular a dye or a photosensitizing drug, such as, in the case of PDT, a vasoconstrictor.

It has been noted that the use of a therapeutic light beam having the aforementioned wavelength feature advantageously and surprisingly made it possible to effectively treat neovascular corneas, without it being necessary to use a drug, as in the case of PDT, or also of reducing the density of blood vessels on the conjunctiva. In addition, in the range of the wavelengths of the invention, the risks for the retina are lower than with the aforementioned lasers of the prior art.

The treatment apparatus is preferably more specifically characterized by one and/or the other of the additional features below, taken alone or in combination with one another:
the source is designed to emit a therapeutic pulsed light beam;
the time of each pulse can be adjusted;
the time of each pulse can be set to a value less than 0.5 s, and preferably at least between 0.1 s and 0.3 s;
the time interval between two pulses is adjustable;
the time interval between two pulses can be set to a value greater than 0.5 s, and preferably to a value greater than or equal to 0.9 s;
the time of emission of the therapeutic light beam is adjustable;
the number of pulses in each emission is adjustable;
the number of pulses in each emission can be set to at least between 50 and 300;
the power of the therapeutic light beam is adjustable;
the power of the therapeutic light beam can be set to at least between 1 W and 5 W;
the power density of the pulses can be set to at least between 30 W/cm² and 300 W/cm²;
the source is a laser source;
the laser source comprises a Raman fiber laser;
the Raman fiber laser includes a pump laser diode, an ytterbium-doped fiber laser, and a Raman converter that is intended to transpose the wavelength of the beam generated by the ytterbium-doped fiber laser.

The treatment method according to the invention preferably has one and/or the other of the following additional characteristics, alone or in combination with one another:
the therapeutic light beam is advantageously a pulsed beam;
the power density (d) of the laser beam at the level of the illuminated site (cornea, limbus or conjunctiva) is preferably between 30 W/cm² and 300 W/cm², and is more preferably on the order of 100 W/cm²;
the pulse fluence is preferably between 1 J/cm² and 300 J/cm²;
the total fluence for each emission is between 6000 J/cm² and 90,000 J/cm², and is more preferably on the order of 30,000 J/cm²;
the time (T) between two successive pulses is greater than 0.5 s, and more specifically greater than or equal to 0.9 s;
the number of pulses (N) in each emission is preferably between 50 and 300 pulses;
the time (t) of each pulse is preferably less than 0.5 s, and more preferably between 0.1 s and 0.3 s;
the operation of lighting of the cornea or the limbus site is repeated a number of times in the case of corneal neovascularization, or of the conjunctiva in the case of the accumulation of vessels on the conjunctiva, preferably with at least one day of rest between each lighting operation.

### DESCRIPTION OF THE FIGURE

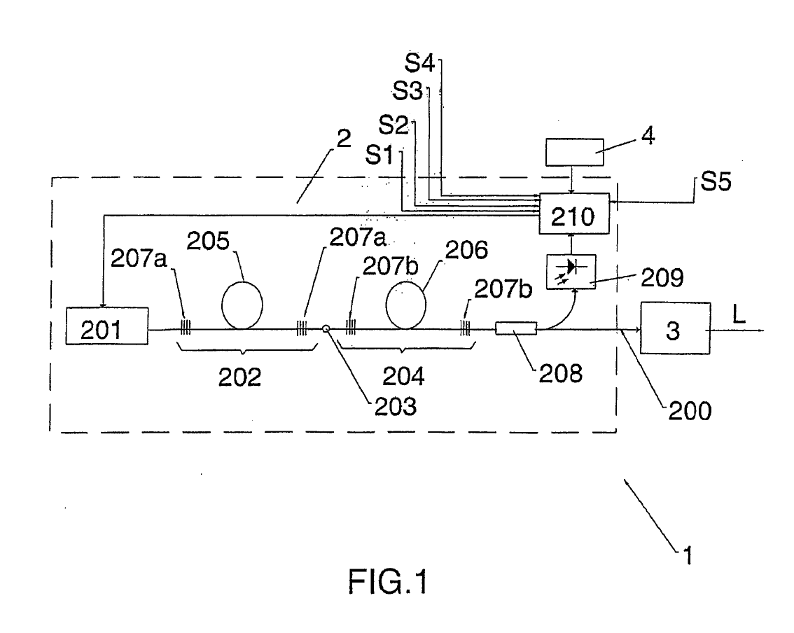

Other characteristics and advantages of the invention become clearer from the following description of a preferred embodiment of a treatment apparatus of the invention and the use thereof, which description is provided by way of a non-limiting example, in reference to the appended FIG. 1 showing a general diagram of an apparatus according to the invention for the treatment of corneal neovascularization or the accumulation of vessels on the conjunctiva.

### DETAILED DESCRIPTION

Apparatus for treatment of corneal neovascularization or the accumulation of vessels on the conjunctiva

In reference to the diagram of the appended FIG. 1, the apparatus 1 for treatment essentially comprises a light source 2 with a fiber output 200, and an adaptation interface 3.

The adaptation interface 3 enables the therapeutic light beam (L) generated at the output 200 by the source 2 to be directed on the zone of the eye that must be treated (cornea, limbus or conjunctiva). This interface 3 can have various known forms.

By way of a non-exhaustive example, the interface 3 is, for example, a hand piece enabling the fiber output of the source 2 to be manipulated by hand, or it can be produced by means of a slit lamp. Examples of hand pieces are described in particular in the patents U.S. Pat. No. 4,900,143 and U.S. Pat. No. 5,346,468 and U.S. Pat. No. 5,951,544. An example of a slit lamp is described in U.S. Pat. No. 5,002,336. In the case of the use of a slit lamp, it preferably and routinely comprises a sighting laser.

Regardless of the adaptation interface 3, the light source 2 is designed to emit, at the output 200, a therapeutic light beam having an emission wavelength between 1.2 μm and 1.3 μm.

This therapeutic light beam is preferably a coherent light beam (laser). Nevertheless, in another embodiment, the therapeutic light beam can be an incoherent light beam, generated by a light source having a sufficient power followed by optical filtering so as to retain only the frequency components in the range of 1.2 μm to 1.3 μm.

In reference to FIG. 1, the light source 2 of the apparatus 1 also comprises means (208, 209, 210, S1, S2, S3, S4, S5) enabling the practitioner to adjust the main beam (L) emission parameters (in particular, power, number of pulses, time of each pulse, time interval between two pulses); these adjustment means will be described below in greater detail.

The apparatus 1 also comprises control means 4, which enable the practitioner to control the activation of the therapeutic light beam according to the emission parameters that have been set. These control means 4 comprise, for example, an action pedal or any other equivalent manual activation means.

When the therapeutic light beam is a laser beam, in its most general sense, the invention is not limited to a specific type of laser source 2, as any laser source allowing for the emission of a laser beam satisfying the aforementioned wavelength condition, and known to a person skilled in the art, can be used. In particular, in a non-exhaustive manner, it is possible to use the following types of laser source:
Raman fiber laser, continuous or pulsed;
Cr: Forsterite (Cr₄₊: Mg₂SIO₄) laser, pulsed or continuous, pumped by a neodymium (Nd)-doped solid or fiber laser, by an ytterbium-doped solid or fiber laser, or diode-pumped;
pulsed or continuous parametric oscillator, pumped by another laser source;
power laser diode;
solid continuous or pulsed Raman converter or laser pumped by another laser source.

Among the lasers mentioned above, a Raman fiber laser is preferably used for the following reasons:
the fiber output of the laser facilitates the transport of the beam to the output 200;
the laser beam generated has a good spectral and spatial quality;
the laser source 2 is advantageously compact;
the laser source 2 is reliable and does not require any maintenance;
this type of laser source provides the best compromise between quality and production cost of the laser.

Preferred embodiment of a Raman fiber laser with a wavelength between 1.2 μm and 1.3 μm

In reference to FIG. 1, the source 2 is a Raman fiber laser and comprises a pump laser diode 201 with a wavelength of 910-930 nm or 970-980 nm, an ytterbium (Yb)-doped fiber laser 202, and a Raman converter 204 that is intended to transpose the wavelength of the beam at the output of the fiber laser 202, so as to obtain a laser beam with a wavelength of 1260-1270 nm.

The ytterbium (Yb)-doped fiber laser 202 consists of a double-coated fiber 205 of which the core is doped with ytterbium and two Bragg gratings 207a at the input and output, which are photoinscribed in the fiber. The output 203 of the fiber of the laser 202 is directly welded to the input of the Raman converter 204.

The Raman converter 204 includes a fiber 206 of which the core is doped with phosphorus and two Bragg gratings 207b at the input and the output, which are set to a wavelength in the range of 1260-1270 nm. This converter 204 makes it possible to perform the transposition of the emission wavelength of the laser 202 in a single step.

In another alternative, it is possible to use a monomode fiber, different from the aforementioned fiber; it is appropriate in this case to adapt the number of steps in the conversion of the Raman 204 converter according to the nature of the fiber, and in particular the type of doping agent used.

It is also possible to replace the Bragg gratings with monomode couplers.

The Raman fiber laser described above in reference to FIG. 1, which allows for the emission of a therapeutic laser beam at a wavelength between 1.2 μm and 1.3 μm, is novel per se, and can therefore advantageously be used in other applications (medical or non-medical), outside of the specific field of the treatment of corneal neovascularization or the accumulation of vessels on the conjunctiva.

In reference to FIG. 1, the power of the laser beam is adjusted via a coupler 208 having a low lock-in rate, and a photodiode 209 connected to electronic control means 210. The electronic control means 210 also receive, at the input, a first continuous set point signal (S1) of which the value is manually set by the practitioner (for example, by means of a potentiometer or the like) and that characterizes the set point power in continuous mode of the laser beam. From this set point value (signal S1), the electronic control means 210 automatically set the power of the laser beam emitted by acting at the output directly on the current of the pump diode 201. The electronic control means 210 thus enable the practitioner to manually set the power of the therapeutic laser beam at a predefined value (set point signal S1).

In addition, the electronic control means 210 receive, at the input, four other continuous set point signals S2, S3, S4 and S5 of which the values are manually set by the practitioner:
the set point signal S2 characterizes, for example, the operation mode (continuous or pulsed);
the set point signal S3 characterizes, for example, in the case of a pulsed mode, the time of each pulse of the therapeutic laser beam;
the set point signal S4 characterizes, for example, in the case of a pulsed mode, time interval between two successive pulses,
the set point signal S5 characterizes the time of emission (or in other words the number of pulses in the case of a pulsed mode) of the therapeutic laser beam, upon each actuation of the control means 4.

The electronic control means 210 thus control the current of the pump diode 201 on the basis of the set point signals S1 to S5 and the signal extracted by the coupler 208 and the photodiode 209, so as to automatically set the physical characteristics of the emitted laser beam (power, mode (pulsed or continuous), emission time, and in the case of a pulsed mode: time of each pulse and time interval between each pulse).

### Treatment Method

The apparatus of the invention is implemented as follows:

Step 1: the practitioner manually sets the emission parameters of the therapeutic laser beam (power, mode (pulsed or continuous), emission time (or number of pulses in the case of a pulsed mode), and in the case of a pulsed mode: time of each pulse and time interval between two pulses).

Step 2: by means of the adaptation interface 3, the practitioner adjusts, in a manner that is very precise and known per se, the spatial position of the laser beam with respect to the site to be illuminated (cornea, limbus or conjunctiva).

Step 3: When the alignment is perfect, the practitioner actuates the control pedal 4, which activates the emission of the therapeutic beam (lighting of the site to be treated) with the predefined emission parameters.

When the targeted site is treated, the practitioner repeats the operations of steps 2 and 3 on another site to be treated, as many times as is necessary to cover the entire surface to be treated. Depending on the case, this surface can be the total surface of the cornea or only a portion of the corneal surface. In the case of corneal neovascularization, the neovessels extend toward the cornea from the limbus; it is therefore also recommended, in order to treat the corneal neovascularization, to illuminate the limbus, in particular at the border with the cornea. In the case of the accumulation of vessels on the conjunctiva, depending on the case, all or some of the surface of the bulbar and palpebral conjunctiva is illuminated.

The aforementioned operations are repeated at a frequency according to the treatment protocol determined on a case-by-case basis by the practitioner.

Comparative tests conducted in the laboratory have made it possible to demonstrate that the use of a pulsed laser beam (L) is preferable to the use of a continuous laser beam, because it makes it possible to reduce the risk of burning the cornea, the limbus of the conjunctiva.

More specifically, the treatment method and the apparatus of the invention preferably have one and/or the other of the technical features below.

The power density (d) of the laser beam at the level of the targeted site (cornea, limbus or conjunctiva) to be treated is preferably between 30 W/cm² and 300 W/cm², and is more preferably on the order of 100 W/cm², with the reminder that the power density (d) is defined by the following formula:

d=P/S

With P representing the pulse power and S representing the surface of the spot formed by the laser beam at the level of the site to be treated.

The pulse fluence is preferably between 1 J/cm² and 30 J/cm². It is noted here that the pulse fluence (F) is defined by the following formula:

F=d×t

in which formula d represents the pulse power density and t represents the pulse duration.

The surface (S) of the spot is dependent on the diameter of the laser beam at the output of the fiber, the “waist” of the beam and the distance between the fiber output of the laser and the site to be treated. For a given waist and diameter of the laser beam, the farther the fiber output of the laser is, the greater the surface of the spot will be, and the lower the power density and the pulse fluence will be.

The total fluence for each emission is preferably between 6000 and 90,000 J/cm², and is more preferably on the order of 30,000 J/cm², with the reminder that the total fluence (FT) for each emission is defined by the following formula:

FT=F×N

where N represents the number of pulses in each emission and F represents the pulse fluence.

The time (T) between two successive pulses must be great enough to prevent any overheating of the tissue (cornea, limbus or conjunctiva). The time (T) between two successive pulses is preferably greater than 0.5 s, and more specifically greater than or equal to 0.9 s.

More specifically, a satisfactory compromise, which makes it possible to comply with the aforementioned fluence values while limiting the treatment time in each emission so as not to immobilize the patient for too long, was obtained with a number of pulses (N) in each emission preferably between 50 and 300 pulses with a time (t) of each pulse between 0.1 s and 0.3 s.

More specifically, the treatment apparatus is preferably characterized by a beam of which the pulse power is between 1 W and 5 W and is more preferably on the order of 3 W, and of which the pulse power density at the output of the apparatus is between 30 W/cm² and 300 W/cm², and more preferably on the order of 100 W/cm².

In a specific example of an embodiment, given purely by way of indication, the treatment apparatus was a fiber laser with a hand piece, and the treatment laser beam generated by the apparatus had a diameter on the order of 2 mm, and was intended to be used by positioning the fiber output at around 10 cm from the site to be illuminated.

The treatment protocol is defined by the practitioner in particular on the basis of the importance of the vessels (density and/or size of the neovessels on the cornea or vessels on the conjunctiva) and also the desired time of immobilization for the patient.

Nevertheless, it should be emphasized that, advantageously, it is possible for the treatment of the invention to cause no harmful adverse effects, and in particular no overheating of the cornea, the limbus or the conjunctiva. It is therefore also desirable to shorten the total time of the treatment protocol by performing, in a single day, a number of successive operations of lighting the cornea, the limbus or the conjunctiva, without being required to provide a day of rest between each operation, as in the aforementioned protocol examples.

The time of the protocol will be dependent on the extent of the proliferation of the neovessels or vessels and the desired result.

In the case of corneal neovascularization, it is possible, depending on the case, to illuminate only zones of the cornea affected by neovessels or zones of the limbus from which these neovessels are extending; in this case, dilation and then hemorrhaging of these neovessels are observed. It is also advantageously possible, in a preventative manner, to illuminate the zones of the cornea not yet affected in a manner visible to the naked eye by neovessels, thereby enabling the propagation of the neovessels to be limited.

However, the invention is not limited to the aforementioned parameters and conditions of use, which are given solely by way of indication.

1. An apparatus for the treatment of corneal neovascularization, or the accumulation of vessels on the conjunctiva in a patient in need thereof, comprising a therapeutic light source that is designed to emit a therapeutic light beam having a wavelength between 1.2 μm and 1.3 μm, wherein said therapeutic light beam is sufficient to treat corneal neovascularization, or the accumulation of vessels on the conjunctiva in said patient.

2. The apparatus according to claim 1, wherein said source is designed to emit a pulsed therapeutic light beam.

3. The apparatus according to claim 2, wherein the time of each pulse is adjustable.

4. The apparatus according to claim 2, wherein the time of each pulse can be set to a value less than 0.5 s.

5. The apparatus according to claim 2, wherein the time interval between two pulses is adjustable.

6. The apparatus according to claim 5, wherein the time interval between two pulses can be set to a value greater than 0.5 s.

7. The apparatus according to claim 2, wherein the time of emission or the number of pulses in each emission of the therapeutic light beam is adjustable.

8. The apparatus according to claim 2, wherein the number of pulses in each emission can be set to at least between 50 and 300.

9. The apparatus according to claim 1, wherein the power of the therapeutic light beam is adjustable.

10. The apparatus according to claim 9, wherein the power of the therapeutic light beam can be set to at least between 1 W and 5 W.

11. The apparatus according to claims 2 or 9, wherein the power density of the pulses can be set to at least between 30 W/cm² and 300 W/cm².

12. The apparatus according to claim 1, wherein the source is a laser source.

13. The apparatus according to claim 12, wherein the laser source comprises a Raman fiber laser.

14. The apparatus according to claim 13, wherein the Raman fiber laser includes a pump laser diode, an ytterbium-doped fiber laser, and a Raman converter that is intended to transpose the wavelength of the beam generated by the ytterbium-doped fiber laser.

15. A method for treatment of corneal neovascularization or the accumulation of vessels on the conjunctiva in a patient in need thereof, comprising illuminating the site of the cornea or limbus to be treated in the case of corneal neovascularization, or the site of the conjunctiva to be treated in the case of the accumulation of vessels on the conjunctiva, with a therapeutic light beam having a wavelength between 1.2 ∥m and 1.3 μm, wherein said therapeutic light beam is sufficient to treat corneal neovascularization or the accumulation of vessels on the conjunctiva in said patient.

16. The treatment method according to claim 15, wherein the therapeutic light beam is a laser beam.

17. The treatment method according to claim 15, wherein the therapeutic light beam is pulsed.

18. The treatment method according to claim 17, wherein the pulse fluence is between 1 J/cm² and 30 J/cm².

19. The treatment method according to claim 17, wherein that the time (T) between two successive pulses is greater than 0.5 s.

20. The treatment method according to claim 17, wherein the time (T) between two successive pulses is greater than or equal to 0.9 s.

21. The treatment method according to claim 17, wherein the number of pulses (N) in each emission is between 50 and 300 pulses.

22. The treatment method according to claim 17, wherein the time (t) of each pulse is less than 0.5 s.

23. The treatment method according to claim 17, wherein in that the time (t) of each pulse is between 0.1 s and 0.3 s.

24. The treatment method according to claim 15, wherein the power density (d) of the therapeutic light beam at the level of the site to be treated is between 30 W/cm² and 300 W/cm².

25. The treatment method according to claim 15, wherein the power density (d) of the therapeutic light beam at the level of the site to be treated is substantially equal to 100 W/cm².

26. The treatment method according to claim 15, wherein the total fluence for each emission is between 6000 J/cm² and 90,000 J/cm².

27. The treatment method according to claim 15, wherein the total fluence for each emission is substantially equal 30,000 J/cm².

28. The treatment method according to claim 15, wherein the operation of lighting the site to be treated is repeated a number of times, with at least one day of rest between each lighting operation.

29. The treatment method according to claim 15, wherein no products are administered to the patient.

30. The apparatus according to claim 4, wherein the time of each pulse can be set to a value between at least 0.1 s and 0.3 s.

31. The apparatus according to claim 6, wherein the time interval between two pulses can be set to a value greater than or equal to 0.9 s.
